(19) United States
(12) Patent Application Publication
(10) Pub. No.: US 2024/0269181 A1
(43) Pub. Date: Aug. 15, 2024

(54) METHOD FOR REGENERATING HUMORAL IMMUNITY SYSTEM AND USE THEREOF

(71) Applicant: GUANGZHOU INSTITUTES OF BIOMEDICINE AND HEALTH CHINESE ACADEMY OF SCIENCES, Guangzhou (CN)

(72) Inventors: Jinyong WANG, Guangzhou (CN); Qi ZHANG, Guangzhou (CN); Bingyan WU, Guangzhou (CN); Fangxiao HU, Guangzhou (CN); Qitong WENG, Guangzhou (CN); Huan PENG, Guangzhou (CN); Yao WANG, Guangzhou (CN); Lijuan LIU, Guangzhou (CN); Xiaofei LIU, Guangzhou (CN); Chengxiang XIA, Guangzhou (CN); Yang GENG, Guangzhou (CN); Hongling WU, Guangzhou (CN)

(21) Appl. No.: 18/562,645

(22) PCT Filed: Apr. 8, 2022
(86) PCT No.: PCT/CN2022/085730

§ 371 Date: Nov. 20, 2023

(30) Foreign Application Priority Data
May 20, 2021 (CN) 202110552087.3
Mar. 10, 2022 (CN) 202210234297.2

(51) Int. Cl. A61K 35/17; A61K 39/00; A61P 37/04; C12N 5/00; C12N 5/0781; C12N 15/85

(52) U.S. Cl. CPC A61K 35/17; A61K 39/4612; A61P 37/04; C12N 5/0018; C12N 5/0635; C12N 15/85

(57) ABSTRACT

A method for regenerating a humoral immunity system. A pluripotent stem cell is used for expressing a RUNX1 gene, a HOXA9 gene and an LHX2 gene to efficiently obtain B cell seeds after in vitro induction differentiation, and after transplantation, a complete humoral immune system can be reconstructed in an animal in which the humoral immune system is missing. According to the method, an antigen-specific antibody immune response can be realized, a specific high-affinity antibody can be generated against an antigen, and immunological memory can be produced. Meanwhile, the reconstructed immune system is safe, and carries no risk of tumorigenicity.

### TECHNICAL FIELD

The present application belongs to the technical field of medical bioengineering, relates to the directional differentiation of pluripotent stem cells and, in particular, relates to a method for regenerating a humoral immunity system and a use thereof, that is, a method for directionally differentiating a pluripotent stem cell into a B cell and a use thereof.

### BACKGROUND

B cells are a core cell component of a humoral immunity system, and a functional defect of the B cells leads to a decline in humoral immunity of a patient and even a severe infection of bacteria, viruses or other pathogenic microorganisms. Therefore, restoring and even enhancing the humoral immunity system through a regeneration means is expected to benefit a large number of patients with abnormal humoral immunity systems.

Pluripotent stem cells (PSCs) are a type of cell that has infinite proliferation potential and potential to differentiate to produce cells of different lineages and is convenient for gene editing and modification. Therefore, the pluripotent stem cells are a hotspot for a cell therapy regenerative medicine research. Inducing pluripotent stem cells derived from reprogramming autologous somatic cells of the patient to differentiate into cells of different lineages can not only avoid the ethical controversy of using embryonic stem cells, but also reduce a risk of allogeneic immune rejection. Therefore, the pluripotent stem cells become an ideal cell development material in the field of regenerative medicine.

How to induce the pluripotent stem cells to differentiate to produce B-lineage seeds and reconstitute the humoral immunity system after transplantation is a hotspot and difficulty to study in the world. So far, no substantial breakthrough has been made, and no case of clinical transformation has been obtained.

A research has shown that after co-culture with stromal cells in vitro, human embryonic stem cells (ESCs) are more easily induced to produce NK cells instead of B cells (see Martin, Colin H et al. Differences in lymphocyte developmental potential between human embryonic stem cell and umbilical cord blood-derived hematopoietic progenitor cells. Blood vol. 112, 7 (2008): 2730-7).

After co-culture with stromal cells in vitro, mouse induced pluripotent stem cells can be induced to produce T cells. However, it is difficult to induce the murine induced pluripotent stem cells to produce B cells (see Wada, Haruka et al. Successful differentiation to T cells, but unsuccessful B-cell generation, from B-cell-derived induced pluripotent stem cells. International immunology vol. 23, 1 (2011): 65-74).

Moreover, research methods about the reconstitution of B cells in vivo are less. A research has shown that after transplantation into immunodeficient mice, pro/pre-B progenitor cells produced by ESCs induced in vitro can produce B1 and B2 cells. However, the B cells produced in the above research are present for a very short time in vivo, and secreted antibodies are not detectable 6 to 8 weeks after the transplantation (see Potocnik, A J et al. Reconstitution of B cell subsets in Rag deficient mice by transplantation of in vitro differentiated embryonic stem cells. Immunology letters vol. 57, 1-3 (1997): 131-7). Another research has reported that ESCs can be induced to produce progenitor cells of B1 cells in vitro and the B1 cells can be reconstituted for a long term after the progenitor cells of the B1 cells are transplanted into immunodeficient mice (see Lin, Yang et al. Long-Term Engraftment of ESC-Derived B-1 Progenitor Cells Supports HSC-Independent Lymphopoiesis. Stem cell reports vol. 12, 3 (2019): 572-583). However, through this method, B2 cells that are more important to an adaptive humoral immune response cannot be obtained.

In addition, in another research, specific transcription factors are expressed in pluripotent stem cells to obtain hematopoietic stem and progenitor cells (HSPCs) having multi-lineage hematopoietic reconstitution capabilities, and after transplantation, cells of multiple hematopoietic lineages including B cells can be produced (see Lu, Yi-Fen et al. Engineered Murine HSCs Reconstitute Multi-lineage Hematopoiesis and Adaptive Immunity. Cell reports vol. 17, 12 (2016): 3178-3192; and Sugimura, Ryohichi et al. Haematopoietic stem and progenitor cells from human pluripotent stem cells. Nature vol. 545, 7655 (2017): 432-438). However, the above research system has the problems of poor stability and relatively low efficiency.

Therefore, an efficient method for inducing pluripotent stem cells to obtain single B-lineage seed cells is urgently needed in the art.

### SUMMARY

The present application provides a method for directionally differentiating a pluripotent stem cell into a B cell and a use thereof. Gene-modified pluripotent stem cells are induced to differentiate in vitro to efficiently obtain B cell seeds, and after transplantation, a complete humoral immunity system can be regenerated in an animal where the humoral immunity system is missing. The method is an efficient method for regenerating the humoral immunity system. An antigen-specific antibody immune response can be achieved, a specific high-affinity antibody can be produced against an antigen, and immunological memory can be produced. The immune system reconstituted through the method is safe, and no risk of tumorigenicity is seen.

In a first aspect, the present application provides an expression vector. The expression vector includes a nucleotide sequence encoding a RUNX1 gene, a nucleotide sequence encoding a HOXA9 gene and a nucleotide sequence encoding an LHX2 gene for achieving the tandem co-expression of the three genes RUNX1, HOXA9 and LHX2.

In the present application, the cDNA sequences of RUNX1, HOXA9 and LHX2 are expressed in tandem in the same vector and integrated into a genome of pluripotent stem cells of a mammal so that host cells stably expressing RUNX1, HOXA9 and LHX2 can be obtained, which is easy to operate and has relatively high efficiency, and the obtained host cells have an ability to differentiate into B cells.

The RUNX1 gene, also known as AML1, is one of the members of a RUNX transcription factor protein family and is the most common target site for a chromosomal translocation in leukemia. RUNX1 is a very critical hematopoietic regulatory transcription factor, which plays an important role in endothelial-to-hematopoietic transition, primitive hematopoiesis, permanent hematopoiesis and lymphopoiesis; and
the RUNX1 gene may be of multiple sources, such as human or murine, wherein the murine RUNX1 gene may be ENSMUSG00000022952, and the human RUNX1 gene may be ENSG00000159216.

The HOXA9 gene, which is a member of a HOX gene family, is a specific transcriptional regulation factor of a coding sequence and plays an important role in embryonic development and hematopoietic regulation. HOXA9 can play an important role in the enhancement and maintenance of HSCs, the endothelial-to-hematopoietic transition and the promotion of lymphogenesis; and
the HOXA9 gene may be of multiple sources, such as human or murine, wherein the murine HOXA9 gene may be ENSMUSG00000038227, and the human HOXA9 gene may be ENSG00000078399.

The LHX2 gene (Lim homeobox 2) is also known as LH-2. As one of the members of a transcription factor family, the LHX2 gene plays a relatively important role in development processes of multiple organs and is expressed in a nervous system at a high level in particular. Moreover, LHX2 plays an important role in embryonic hematopoiesis and erythropoiesis and can promote the immortalization of hematopoietic stem and progenitor cells. In addition, LHX2 is found to be expressed in a pre-B cell line.

The LHX2 gene may be of multiple sources, such as human or murine, wherein the murine LHX2 gene may be ENSMUSG00000000247, and the human LHX2 gene may be ENSG00000106689.

A main reason of the combination of the three genes is that RUNX1 can promote the pluripotent stem cells to differentiate into hemogenic endothelial cells, RUNX1 and HOXA9 can promote the lymphopoiesis and LHX2 further promotes the differentiation into a B lineage. Compared with other differentiation-related genes, such as using RUNX1, LMO2 and MEIS1 at the same time, the combination cannot normally produce a hematopoietic clone in a late process of co-culturing induced hemogenic endothelial cells with OP9-DL1.

In a second aspect, the present application provides a gene-edited pluripotent stem cell host cell. The host cell includes the expression vector according to the first aspect.

Preferably, the host cell is a pluripotent stem cell including an induced pluripotent stem cell and/or an embryonic pluripotent stem cell line.

Preferably, the pluripotent stem cell includes a gene-edited induced pluripotent stem cell and/or embryonic pluripotent stem cell line.

In a third aspect, the present application provides a method for regenerating a humoral immunity system, that is, a method for directionally differentiating a pluripotent stem cell into a B cell. The method includes the following steps:
(1) integrating the expression vector according to the first aspect, that is, the expression vector where the three genes RUNX1, HOXA9 and LHX2 are in tandem, into a pluripotent stem cell and performing resistance cloning screening;
(2) directionally differentiating the pluripotent stem cell obtained in step (1) into an induced hemogenic endothelial cell (iHEC);
(3) co-culturing the induced hemogenic endothelial cell in step (2) with a bone marrow stromal cell to obtain a hematopoietic progenitor cell having B-lineage differentiation potential, namely, a B-lineage seed cell; and
(4) transplanting the B-lineage seed cell in step (3) and differentiating in vivo to produce a B cell.

In the present application, the directional differentiation is performed on the pluripotent stem cell line where RUNX1, HOXA9 and LHX2 are co-expressed to obtain the induced hemogenic endothelial cell, which is co-cultured with the bone marrow stromal cell to obtain the B-lineage seed cell, and after the differentiation, the B cell with a normal function is obtained, including all types of mature cell and having no risk of tumorigenesis.

Preferably, the expression vector where RUNX1, HOXA9 and LHX2 are in tandem in step (1) may be integrated into any safe site, and an insertion site enables an inserted gene to be stably expressed. In the present application, preferably, the genes expressed in tandem are integrated into a ROSA26 site, an AAVS1 site, a CCR5 site, an H11 site, a COL1A1 site or a TIGRE site of the pluripotent stem cell.

Preferably, the pluripotent stem cell in step (1) is a gene-edited induced pluripotent stem cell and/or embryonic pluripotent stem cell line.

Preferably, a method for the integration in step (1) includes any one or a combination of at least two of homologous recombination, CRISPR/Cas9, TALEN, transfection or viral infection, preferably the homologous recombination.

Preferably, hygromycin B is used for the resistance screening in step (1) to obtain a main clonal stem cell line. Other resistance screening strategies such as chloramphenicol, geneticin (G-418), blasticidin and mycophenolic acid may also be used to obtain the main clonal stem cell line.

Preferably, a method for the directional differentiation in step (2) is as follows: culturing the pluripotent stem cell using a DO medium, a D2.5 medium and a D6 medium in sequence to obtain the induced hemogenic endothelial cell.

Preferably, the DO medium is a basal differentiation medium containing 3 to 8 ng/mL bone morphogenetic protein 4 (BMP4). The concentration of the bone morphogenetic protein 4 may be, for example, 3 ng/mL, 4 ng/mL, 5 ng/mL, 6 ng/mL, 7 ng/mL or 8 ng/mL, preferably 5 ng/mL.

Preferably, the D2.5 medium is a basal differentiation medium containing 3 to 8 ng/mL (which may be, for example, 3 ng/mL, 4 ng/mL, 5 ng/mL, 6 ng/mL, 7 ng/mL or 8 ng/mL, preferably 5 ng/mL) BMP4 and 3 to 8 ng/mL (which may be, for example, 3 ng/mL, 4 ng/mL, 5 ng/mL, 6 ng/mL, 7 ng/mL or 8 ng/mL, preferably 5 ng/mL) vascular endothelial growth factor (VEGF).

Preferably, the D6 medium is a basal differentiation medium containing 10 to 30 ng/mL interleukin 3 (IL3), 10 to 30 ng/mL interleukin 6 (IL6), 10 to 30 ng/mL stem cell factor (SCF), 10 to 30 ng/mL FMS-like tyrosine kinase 3 ligand (Flt3L) and 1 to 2 μg/mL doxycycline (Dox).

The concentration of the IL3 may be, for example, 10 ng/mL, 15 ng/mL, 18 ng/mL, 22 ng/mL, 25 ng/mL or 30 ng/mL, preferably 20 ng/mL. The concentration of the IL6 may be, for example, 10 ng/mL, 15 ng/mL, 18 ng/mL, 22 ng/mL, 25 ng/mL or 30 ng/mL, preferably 20 ng/mL. The concentration of the SCF may be, for example, 10 ng/mL, 15 ng/mL, 18 ng/mL, 22 ng/mL, 25 ng/mL or 30 ng/mL, preferably 20 ng/mL. The concentration of the Flt3L may be, for example, 10 ng/mL, 15 ng/mL, 18 ng/mL, 22 ng/mL, 25 ng/mL or 30 ng/mL, preferably 20 ng/mL. The concentration of the doxycycline may be, for example, 1 μg/mL, 1.2 μg/mL, 1.4 μg/mL, 1.5 μg/mL, 1.6 μg/mL, 1.8 μg/mL or 2 μg/mL, preferably 1 μg/mL.

Components of the DO medium, the D2.5 medium and the D6 medium are shown in the following Table 1:

TABLE 1

| Component | D0 Medium | D2.5 Medium | D6 Medium |
|---|---|---|---|
| basal differentiation | main component | | |
| medium | | | |
| bone morphogenetic | 3 to 8 ng/ml | 3 to 8 ng/ml | — |
| protein 4 | | | |
| vascular endothelial | — | 3 to 8 ng/ml | — |
| growth factor | | | |
| interleukin 3 | — | — | 10 to 30 ng/ml |
| interleukin 6 | — | — | 10 to 30 ng/ml |
| stem cell factor | — | — | 10 to 30 ng/ml |
| FMS-like tyrosine | — | — | 10 to 30 ng/ml |
| kinase 3 ligand | | | |
| doxycycline | — | — | 1 to 2 μg/mL |

Preferably, the basal differentiation medium is an IMDM medium containing 10% to 20% fetal bovine serum (“%” denotes volume fraction), 180 to 220 μg/mL iron-saturated transferrin, 4×10⁻⁴ to 5×10⁻⁴ M thioglycerol, 1 to 3 mM GlutaMAX™-I additive and 30 to 70 μg/mL ascorbic acid;
wherein, the concentration of the fetal bovine serum may be, for example, 10%, 12%, 14%, 16%, 18% or 20%, preferably 15%; the concentration of the iron-saturated transferrin may be, for example, 180 μg/mL, 190 μg/mL, 210 μg/mL or 220 μg/mL, preferably 200 μg/mL; the concentration of the thioglycerol may be, for example, 4×10⁻⁴ M, 4.2×10⁻⁴ M, 4.4×10⁻⁴ M, 4.8×10⁻⁴ M or 5×10⁻⁴ M, preferably 4.5×10⁻⁴ M; the concentration of the GlutaMAX™-I additive may be, for example, 1 mM, 1.4 mM, 1.8 mM, 2.2 mM, 2.4 mM, 2.6 mM, 2.8 mM or 3 mM, preferably 2 mM; and the concentration of the ascorbic acid may be, for example, 30 μg/mL, 35 μg/mL, 40 μg/mL, 45 μg/mL, 55 μg/mL, 60 μg/mL, 65 μg/mL or 70 μg/mL, preferably 50 μg/mL.

In the present application, by changing the added substance in the medium, the inventors design and optimize a directional hematopoietic differentiation system to induce the hematopoietic differentiation of the pluripotent stem cell into the induced hemogenic endothelial cell, and the induced hemogenic endothelial cell is further co-cultured with the mouse bone marrow stromal cell to obtain the B-lineage seed cell.

Preferably, the bone marrow stromal cell in step (3) includes any one or a combination of at least two of an OP9-DL1 cell, an OP9-DL4 cell, an OP9 cell, an MS5 cell, an MS5-DL1 cell, an MS5-DL4 cell, an HS-5 cell, an HS-5-DL1 cell, an HS-5-DL4 cell, an MSC cell, an MSC-DL1 cell or an MSC-DL4 cell. Any one or a combination of at least two of other stromal cells being of sources such as bone marrow, thymus, lymph node, liver and spleen tissues or bone marrow stromal cells modified to express DL1 or DL4 may also be selected.

The above cell lines also have an alias DLL1 when carrying DL1 and an alias DLL4 when carrying DL4, all of which are the same corresponding cell lines.

Preferably, doxycycline (Dox) is used for induction in a process of the co-culture in step (3).

Expression elements designed according to other induction principles may also be used for corresponding drug induction, such as tamoxifen (4-OHT).

Preferably, a method for the co-culture in step (3) is as follows: co-culturing the induced hemogenic endothelial cell with the OP9-DL1 cell using a D11 medium to obtain the B-lineage seed cell.

Preferably, components of the D11 medium are shown in the following Table 2:

TABLE 2

| Component | D11 Medium | |
|---|---|---|
| α-MEM medium | main component | |
| interleukin 3 | 10 to 30 | ng/ml |
| stem cell factor | 10 to 30 | ng/ml |
| FMS-like tyrosine kinase 3 ligand | 10 to 30 | ng/mL |
| doxycycline | 1 to 2 | μg/mL |
| fetal bovine serum | 10 to 20% | |
| iron-saturated transferrin | 180 to 220 | μg/mL |
| thioglycerol | 4.5 × 10⁻⁴M | |
| GlutaMAX ™-I additive | 1 to 3 | mM |
| ascorbic acid | 30 to 70 | μg/mL |

The concentration of the IL3 may be, for example, 10 ng/mL, 15 ng/mL, 18 ng/mL, 22 ng/mL, 25 ng/mL or 30 ng/mL, preferably 20 ng/mL. The concentration of the SCF may be, for example, 10 ng/mL, 15 ng/mL, 18 ng/mL, 22 ng/mL, 25 ng/mL or 30 ng/mL, preferably 20 ng/mL. The concentration of the Flt3L may be, for example, 10 ng/mL, 15 ng/mL, 18 ng/mL, 22 ng/mL, 25 ng/mL or 30 ng/mL, preferably 20 ng/mL. The concentration of the doxycycline may be, for example, 1 μg/mL, 1.2 μg/mL, 1.4 μg/mL, 1.5 μg/mL, 1.6 μg/mL, 1.8 μg/mL or 2 μg/mL, preferably 1 μg/mL. The concentration of the fetal bovine serum may be, for example, 10%, 12%, 14%, 16%, 18% r 20%, preferably 15%. The concentration of the iron-saturated transferrin may be, for example, 180 μg/mL, 190 μg/mL, 210 μg/mL or 220 μg/mL, preferably 200 μg/mL. The concentration of the thioglycerol may be, for example, 4×10⁻⁴ M, 4.2×10⁻⁴ M, 4.4×10⁻⁴ M, 4.8×10⁻⁴ M or 5×10⁻⁴ M, preferably 4.5×10⁻⁴ M. The concentration of the GlutaMAX™-I additive may be, for example, 1 mM, 1.4 mM, 1.8 mM, 2.2 mM, 2.4 mM, 2.6 mM, 2.8 mM or 3 mM, preferably 2 mM. The concentration of the ascorbic acid may be, for example, 30 μg/mL, 35 μg/mL, 40 μg/mL, 45 μg/mL, 55 μg/mL, 60 μg/mL, 65 μg/mL or 70 g/mL, preferably 50 μg/mL.

It is to be noted that the main medium of the D11 medium may be an u-MEM medium or an IMDM medium. Since the bone marrow stromal cell used in an experimental process of the present application is OP9-DL1, the medium is preferably the a-MEM medium.

Preferably, the B cell produced through the differentiation in step (4) includes a B220⁺ B cell and/or a CD19⁺ B cell.

Preferably, the B cell produced through the differentiation includes any one or a combination of at least two of a pro-B cell, a pre-B cell, a B1 cell, a B2 cell or a plasma cell.

Preferably, the B1 cell includes a B1a cell and/or a B1b cell.

Preferably, the B2 cell is a follicular B (FO B) cell and/or a marginal zone B (MZ B) cell.

As a preferred technical solution of the present application, the present application provides a method for directionally differentiating a pluripotent stem cell into a B cell. The method includes the following steps:
(1) integrating the expression vector where RUNX1, HOXA9 and LHX2 are in tandem into the ROSA26 site of the pluripotent stem cell through gene recombination and performing the resistance screening with hygromycin B;
(2) culturing the pluripotent stem cell in step (1) using the DO medium, the D2.5 medium and the D6 medium in sequence and directionally differentiating into the induced hemogenic endothelial cell on days 8 to 12;
(3) co-culturing the induced hemogenic endothelial cell in step (2) with the OP9-DL1 cell using the D11 medium for 8 to 21 days and inducing with doxycycline to obtain the B-lineage seed cell; and
(4) transferring the B-lineage seed cell in step (3) to an animal model and differentiating to produce the B cell, where the B cell includes any one or a combination of at least two of the pro-B cell, the pre-B cell, the B1 cell, the B2 cell or the plasma cell.

Preferably, the directional differentiation into the induced hemogenic endothelial cell in step (2) is on day 11.

Preferably, the co-culture in step (3) is performed for 10 days.

In a fourth aspect, the present application provides a B-lineage seed cell or B cell prepared through the method according to the third aspect.

In a fifth aspect, the present application provides a pharmaceutical composition. The pharmaceutical composition includes any one or a combination of at least two of the expression vector according to the first aspect, the host cell according to the second aspect or the B-lineage seed cell or B cell according to the fourth aspect.

Preferably, the pharmaceutical composition further includes a pharmaceutically acceptable adjuvant, where the pharmaceutically acceptable adjuvant includes any one or a combination of at least two of a carrier, an excipient or a diluent.

In a sixth aspect, the present application further provides a use of the pharmaceutical composition according to the fifth aspect to preparation of a drug for enhancing an immune response, a drug for preventing and/or treating a disease, a drug for a B cell immunotherapy for treating a tumor, a B cell vaccine or a drug for a cell therapy that a B cell secretes a therapeutic protein.

Preferably, the drug for enhancing the immune response includes a drug for enhancing a B cell immune response and/or a T cell immune response.

Preferably, the drug for preventing and/or treating the disease includes a drug for preventing and/or treating a B cell immunodeficiency, an infectious disease and a tumor.

Preferably, the drug for the cell therapy that the B cell secretes the therapeutic protein includes a drug for preventing and/or treating an autoimmune disease and a genetically inherited disease.

Preferably, the therapeutic protein secreted by the B cell includes an antibody.

Preferably, the genetically inherited disease includes any one or a combination of at least two of hemophilia, lysosomal storage disease, hypophosphatasia or phenylketonuria.

In the present application, the pharmaceutical composition can be used for: (1) the enhancement of the immune response, especially the enhancement of the B cell immune response and/or the T cell immune response; (2) the prevention and/or treatment of the disease, preferably for the prevention and/or treatment of the B cell immunodeficiency, the infectious disease and the tumor; (3) the development and preparation of the B cell vaccine; and (4) the cell therapy that the B cell secretes the therapeutic protein, preferably for the prevention and/or treatment of the autoimmune disease and the genetically inherited disease.

Compared with the prior art, the present application has the beneficial effects described below.
(1) In the present application, the vector where exogenous RUNX1, HOXA9 and LHX2 are co-expressed is introduced into the pluripotent stem cell to successfully constitute the induced pluripotent stem cell where exogenous RUNX1, HOXA9 and LHX2 are co-expressed. The pluripotent stem cell has the ability to differentiate into the B cell and can be used for preparing the drugs for enhancing the immune effect, preventing and/or treating the immunodeficiency, preventing and/or treating the infectious disease and preventing and/or treating the tumor, preparing the B cell vaccine and preparing the drug for the cell therapy that the B cell secretes the therapeutic protein.
(2) In the present application, the directional differentiation system and the co-culture method are used for directionally differentiating the pluripotent stem cell into the B-lineage seed cell, and after the transplantation, the B-lineage seed cell can differentiate in vivo to produce the B cell and can be used for preparing the drugs for enhancing the immune effect, preventing and/or treating the immunodeficiency, preventing and/or treating the infectious disease and preventing and/or treating the tumor, preparing the B cell vaccine and preparing the drug for the cell therapy that the B cell secretes the therapeutic protein.
(3) The pluripotent stem cell-derived B cell obtained through the method of the present application has the normal function and no risk of tumorigenesis, can be used for preparing multiple drugs and has a broad application prospect.

### BRIEF DESCRIPTION OF DRAWINGS

FIG. 1 (A) is a schematic diagram illustrating the site-specific knock-in of an inducible expression system at a ROSA26 site of pluripotent stem cells.

FIG. 1 (B) includes a bright field image (left) and fluorescence image (right) (scale 200 μm) of iRUNX1-p2a-HOXA9-t2a-LHX2 pluripotent stem cells obtained through resistance screening with hygromycin B.

FIG. 1 (C) illustrates relative expression levels of RUNX1 (left), HOXA9 (middle) and LHX2 (right) 24 hours after treatment with doxycycline.

FIG. 2 (A) is a schematic diagram of a directional induced differentiation system of embryoid bodies for inducing the iRUNX1-p2a-HOXA9-t2a-LHX2 pluripotent stem cells to directionally differentiate into induced hemogenic endothelial cells (iHECs).

FIG. 2 (B) includes an image (left) (scale 400 μm) illustrating the differentiation morphology of embryoid body (EB) cells obtained after the iRUNX1-p2a-HOXA9-t2a-LHX2 pluripotent stem cells are induced to directionally differentiate to day 11 and an image (right) (scale 400 μm) illustrating hematopoiesis-related cell colonies obtained through the differentiation of the embryoid bodies (EBs).

FIG. 2 (C) is a diagram illustrating flow cytometry results of sorting the induced hemogenic endothelial cells through a flow cytometry sorting strategy (CD31⁺, CD41⁺, CD45⁻, c-Kit⁺ and CD201⁺).

FIG. 3 (A) is a schematic diagram illustrating the co-culture of the sorted induced hemogenic endothelial cells with OP9-DL1 cells.

FIG. 3 (B) is a light field image (scale 400 μm) of a cobblestone-like forming region of hematopoietic cells observed under a microscope 10 days after the induced hemogenic endothelial cells are co-cultured with the OP9-DL1 cells.

FIG. 3 (C) is a diagram illustrating flow cytometry detection results of immunophenotyping of hematopoietic progenitor cells 10 days after the induced hemogenic endothelial cells are co-cultured with the OP9-DL1 cells.

FIG. 4 (A) is a schematic diagram of a transplantation strategy after the co-culture for obtaining B cells using an in vivo microenvironment.

FIG. 4 (B) is a diagram illustrating flow cytometry detection results of blood cells in peripheral blood, bone marrow, spleens and lymph nodes of recipient mice 6 weeks after the transplantation.

FIG. 4 (C) is a schematic diagram of a PCR amplification position of a genome of pluripotent stem cell-derived blood cells.

FIG. 4 (D) is a diagram illustrating the electrophoresis detection of the PCR amplification of the genome of the pluripotent stem cell-derived blood cells; in the figure, lane M denotes DNA Marker, lane 1 denotes a plasmid, lane 2 denotes mouse lymph node (LN) cells, lane 3 denotes mouse spleen (SP) cells, lane 4 denotes mouse bone marrow (BM) cells, and lane 5 denotes a blank control.

FIG. 4 (E) is a diagram illustrating results of the sequencing identification of the genome of the pluripotent stem cell-derived blood cells.

FIG. 4 (F) illustrates contents of immunoglobulin in serums of unimmunized recipient mice (iB mice) detected through ELISA after the transplantation.

FIG. 5 (A) is a diagram illustrating flow cytometry analysis results of pluripotent stem cell-derived B progenitor cells (pro/pre-B), immature B cells and mature B cells in the bone marrow 2 weeks after the transplantation into the recipient mice.

FIG. 5 (B) is a diagram illustrating flow cytometry analysis results of pluripotent stem cell-derived B1 (including Bla and BIb) and B2 (including FO B and MZ B) cell populations in the spleen, the lymph nodes and the peritoneal cavities 4 weeks after the transplantation into the recipient mice.

FIG. 5 (C) illustrates an analysis on the diversity of heavy chains and light chains of B cell receptors (BCRs) of pluripotent stem cell-derived naive follicular B (FO B) cells in the spleens 4 weeks after the transplantation into the recipient mice (iB mice).

FIG. 6 (A) is a diagram illustrating ELISA detection results of antigen-specific (anti-NP) IgM (left) and IgG3 (right) in serums after recipient mice (iB mice) were immunized with T-independent-1 antigens (NP-LPS).

FIG. 6 (B) is a diagram illustrating ELISA detection results of antigen-specific (anti-NP) IgM (left) and IgG3 (right) in the serums after the recipient mice (the iB mice) were immunized with T-independent-2 antigens (NP-AECM-FICOLL).

FIG. 6 (C) includes diagrams illustrating ELISA detection results of antigen-specific (anti-NP) IgM (FIG. 1) and IgG1 (FIG. II and FIG. III) in the serums after the recipient mice (the iB mice) were immunized with T cell-dependent antigens (NP-CGG) the first time and diagrams illustrating ELISA detection results of antigen-specific (anti-NP) IgG1 (FIG. IV and FIG. V) in the serums after the recipient mice (the iB mice) were immunized the second time (the second antigen stimulation is performed on day 111 after the first immunization).

FIG. 7 (A) illustrates flow cytometry detection results of pluripotent stem cell-derived plasma cells and antigen-specific germinal center B (NP-specific GC B) cells in spleens on day 14 after the recipient mice (the iB mice) were immunized with the T cell-dependent antigens (NP-CGG).

FIG. 7 (B) illustrates flow cytometry detection results of IgM⁺ memory B cells and IgG1⁺ memory B cells in the spleens on day 14 after the recipient mice (the iB mice) were immunized with the T cell-dependent antigens (NP-CGG).

FIG. 7 (C) is a diagram illustrating flow cytometry results of long-lived plasma cells in bone marrow of the recipient mice (the iB mice) on day 21 after the first antigen stimulation and on day 17 after the second antigen stimulation (the second antigen stimulation is performed on day 111 after the first antigen stimulation).

### DETAILED DESCRIPTION

Technical solutions of the present application are further described below through specific examples in conjunction with drawings. However, the following examples are only simple examples of the present application and do not represent or limit the protection scope of the present application. The protection scope of the present application is subject to the claims.

In the following examples, unless otherwise specified, the reagents and consumables used are purchased from conventional reagent manufacturers in the art; unless otherwise specified, the experimental methods and technical means used are conventional methods and means in the art.

### Example 1 Preparation of Vectors and Pluripotent Stem Cells Expressing RUNX1, HOXA9 and LHX2 Genes

In this example, the site-specific knock-in of an inducible expression sequence was performed at a ROSA26 site of pluripotent stem cells through an electrotransformation method in conjunction with gene recombination, the expression system used p2a and t2a sequences so that cDNA sequences of RUNX1 (CCDS28339.1), HOXA9 (CCDS20146.1) and LHX2 (CCDS16008.1) were in tandem, and doxycycline (Dox) was used for inducing the expression of the genes.

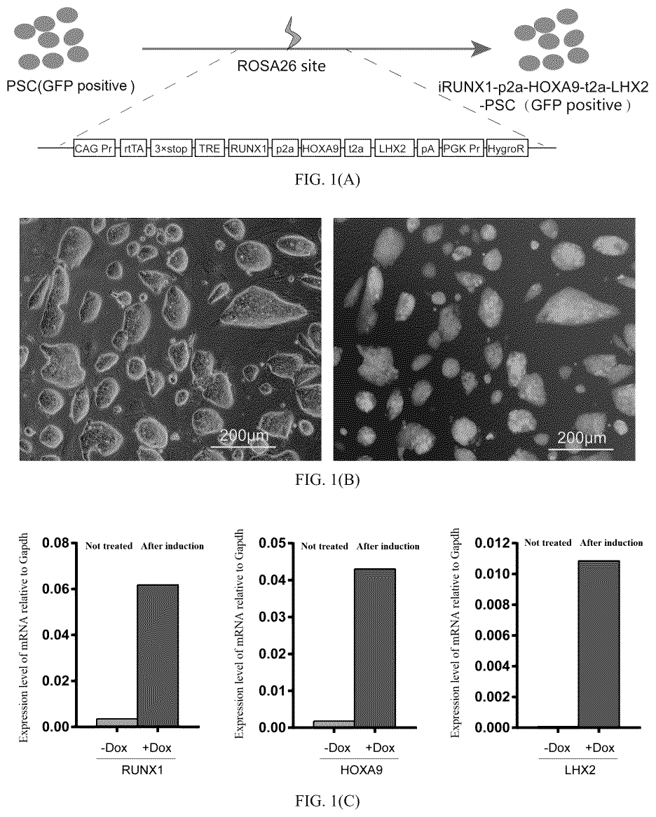

As shown in FIG. 1(A), the knock-in sequence includes an iRUNX1-p2a-HOXA9-t2a-LHX2 tandem sequence and a hygromycin B resistance gene (HygroR) sequence for resistance screening.

To successfully obtain homologous recombinant pluripotent stem cells, a pluripotent stem cell medium containing hygromycin B (150 μg/mL) was added after electrotransformation for 20 hours, and the medium was changed every day. After screening with hygromycin B for 10 days, single clones were picked under a microscope to a 12-well plate laid with mouse embryonic fibroblasts (MEFs) in advance, and one pluripotent stem cell clone was placed in each well and cultured using a hygromycin-free medium.

After clone groups were adhered to cell layers of the MEFs, the media were changed every day. After 3 days, the clone groups were digested with 0.25% trypsin and passaged to the 12-well plate. The cell morphology is shown in FIG. 1 (B), where the clone groups are in a logarithmic growth stage with neat and clear edges and apparently demarcated with the cell layers of the MEFs, and no differentiation occurred. Passage, amplification and cryopreservation were performed according to a cell state and a growth density.

Total mRNA of the iRUNX1-p2a-HOXA9-t2a-LHX2 pluripotent stem cells 24 hours after the treatment with Dox (a group without the addition of Dox was used as a control group) was extracted, and expression levels of mRNA of RUNX1, HOXA9 and LHX2 were detected through Q-PCR. FIG. 1 (C) indicates that the addition of Dox can induce the expression of RUNX1, HOXA9 and LHX2.

### Example 2 Induction of Differentiation of Pluripotent Stem Cells into Induced Hemogenic Endothelial Cells

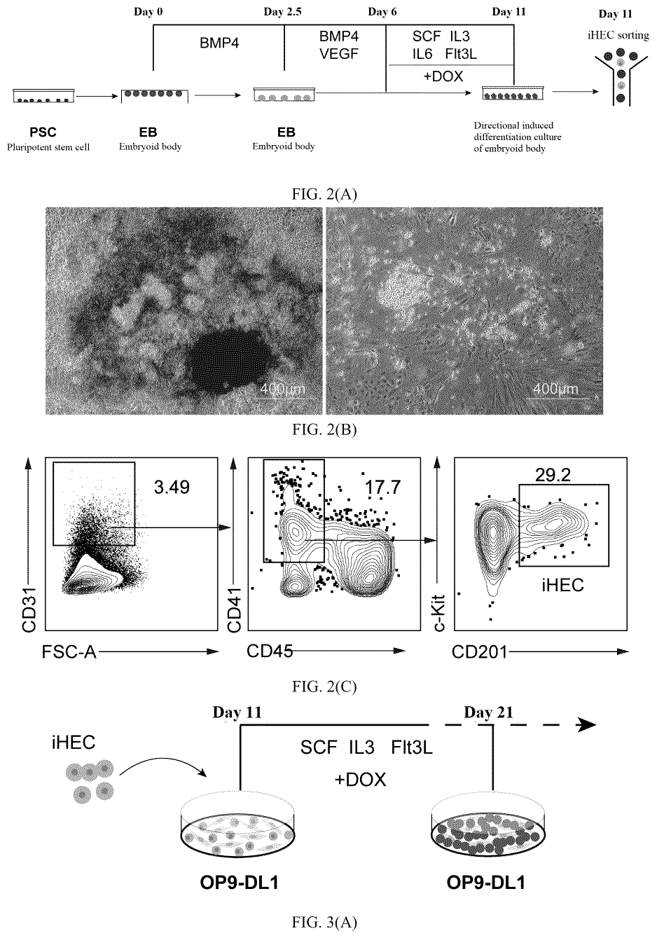

To induce the differentiation of the pluripotent stem cells into the induced hemogenic endothelial cells, a directional induced differentiation system of embryoid bodies shown in FIG. 2 (A) was used.

Formulations for all media in the directional induced differentiation system are as follows: basal differentiation medium: an IMDM medium containing 15% fetal bovine serum, 200 μg/mL iron-saturated transferrin, 4.5×10⁻⁴ M thioglycerol, 2 mM GlutaMAXT™-I additive and 50 μg/mL ascorbic acid;
D0 medium: a basal differentiation medium containing 5 ng/mL bone morphogenetic protein 4;
D2.5 medium: a basal differentiation medium containing 5 ng/mL bone morphogenetic protein 4 and 5 ng/mL vascular endothelial growth factor; and
D6 medium: a basal differentiation medium containing 20 ng/mL recombinant mouse interleukin 3, 20 ng/mL recombinant mouse interleukin 6, 20 ng/mL recombinant mouse stem cell factor, 20 ng/mL human FMS-like tyrosine kinase 3 ligand and 1 μg/mL doxycycline.

Specific steps are described below.
(1) 1 mL gelatin having a concentration of 0.1% was laid in a 6-well plate 40 min in advance for further use. The pluripotent stem cells were digested into single cells using 0.05% trypsin, and after centrifugation, the pluripotent stem cells were resuspended. Excess gelatin was absorbed, and a pluripotent stem cell suspension was transferred to gelatin-coated wells and placed in an incubator for 40 min to remove the MEF cells. Suspended cells were collected, centrifuged at 250 g for 5 min and washed once with DPBS.
(2) The cells were resuspended using the DO medium and counted, and a cell concentration was adjusted to 1×10⁵ cells/mL. 5 to 10 mL cell suspension was added to a tilted 10 cm dish, and 20 μL cell suspension was sucked and added to a 15 cm Petri dish to suspend the embryoid bodies (EBs), where a single EB was 20 μL (approximately 2000 cells). Then, the Petri dish was inverted, a 10 cm Petri dish lid was placed at a bottom of the Petri dish, and 5 to 6 mL cell culture water was added to the lid. The Petri dish was cultured in the incubator for 2.5 days at 37° C.
(3) 1 mL gelatin having a concentration of 0.10% was laid in a 6-well plate 40 min in advance for further use. The EBs were collected in a centrifuge tube using a Pasteur pipette, and the bottom of the dish was washed with DPBS. After the EBs were naturally settled, supernatant was carefully absorbed, and the supernatant may also be removed through low-speed centrifugation at 90 g for 5 min. After the EBs were resuspended using the D2.5 medium, excess gelatin was absorbed, and the EBs were transferred to a gelatin-coated 6-well plate and cultured for 12 hours to observe whether the EBs were contaminated.
(4) Then, the medium was changed on D4, and the culture was continued for two days, where the medium used was the D2.5 medium.
(5) The medium was replaced with the D6 medium and cultured for one day. Then, the medium was changed every other day, where the medium used was the D6 medium.

In a culture process, the embryoid bodies gradually diffused and migrated to peripheries to form mesodermal cells. As shown in FIG. 2 (B), on day 11, an apparent circle of differentiated cells can be seen at the peripheries of the centers of the iRUNX1-p2a-HOXA9-t2a-LHX2 embryoid bodies (left), and apparent hematopoietic clusters can be seen around the embryoid bodies (right).

On day 11 of the induced differentiation and culture of the embryoid bodies, the induced hemogenic endothelial cells were sorted using a flow cytometer through a sorting strategy (CD31⁺, CD41⁺, CD45⁻, c-Kit⁺ and CD201⁺) shown in FIG. 2 (C).

### Example 3 Co-culture of Induced Hemogenic Endothelial Cells with OP9-DL1 Stromal Cells

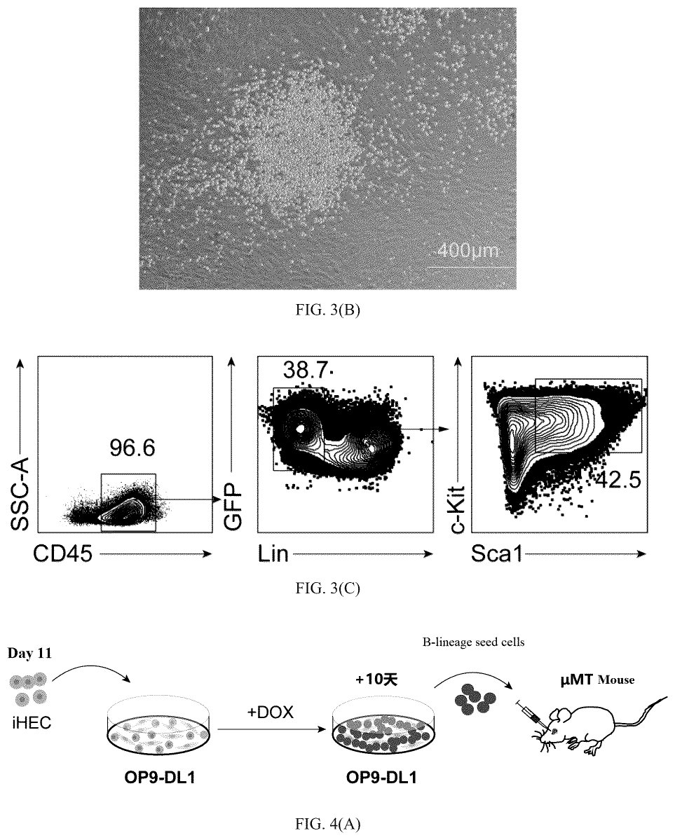

To further induce the induced hemogenic endothelial cells to differentiate to obtain B-lineage seed cells, as shown in FIG. 3 (A), the induced hemogenic endothelial cells obtained through the sorting were co-cultured with the OP9-DL1 stromal cells in this example.

Specific steps are described below.
(1) The OP9-DL1 cells were revived 4 days in advance and passaged in time according to a cell growth state to avoid the aging of the cells due to overgrowth.
(2) The cells were passaged on the day before use, and 20,000 cells were re-laid in each well (a 12-well plate) and used on the next day.

A co-culture medium was a D11 medium, which was a-MEM medium containing 20 ng/mL recombinant mouse interleukin 3, 20 ng/mL recombinant mouse stem cell factor, 20 ng/mL human FMS-like tyrosine kinase 3 ligand, 1 μg/mL Dox, 15% fetal bovine serum, 200 μg/mL iron-saturated transferrin, 4.5×10⁻⁴ M thioglycerol, 2 mM GlutaMAX™-I additive and 50 μg/mL ascorbic acid.

FIG. 3 (B) shows that after the iRUNX1-p2a-HOXA9-t2a-LHX2 pluripotent stem cell-derived induced hemogenic endothelial cells were co-cultured with the stromal cells OP9-DL1 for 10 days, highly uniform small, round and bright hematopoietic cells were formed on the stromal cells OP9-DL1.

FIG. 3 (C) shows that after the iRUNX1-p2a-HOXA9-t2a-LHX2 pluripotent stem cell-derived induced hemogenic endothelial cells were co-cultured with the stromal cells OP9-DL1 for 10 days, the generated hematopoietic cells exhibited immunophenotyping of hematopoietic progenitor cells: LSK (Lin⁻c-Kit⁺Scal⁺).

### Example 4 Transplantation after Co-culture for In Vivo B Lineage Regeneration

To obtain the B cells using an in vivo microenvironment, a transplantation strategy after the co-culture was further designed in this example.

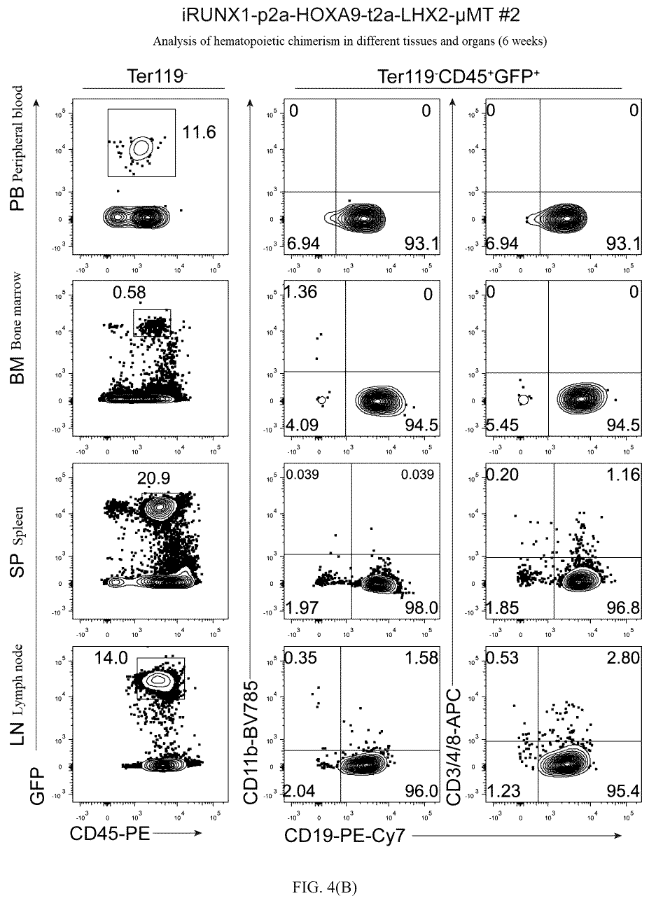

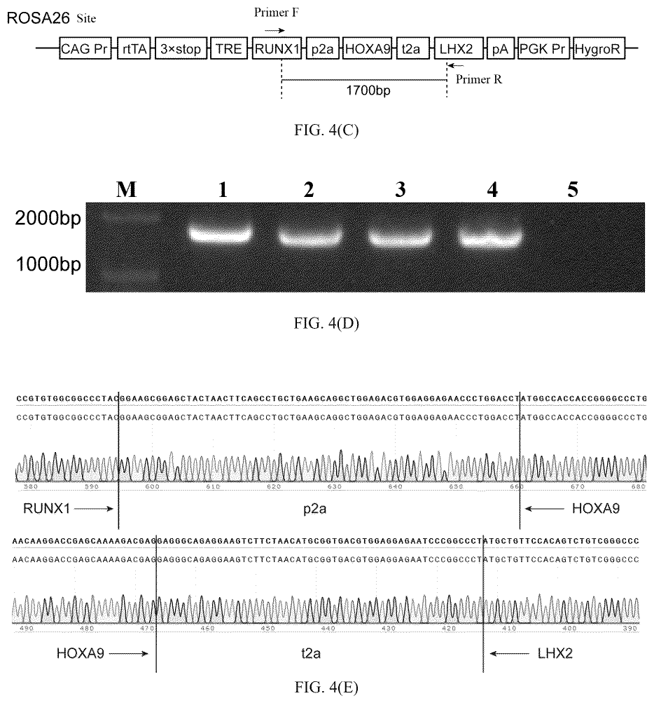

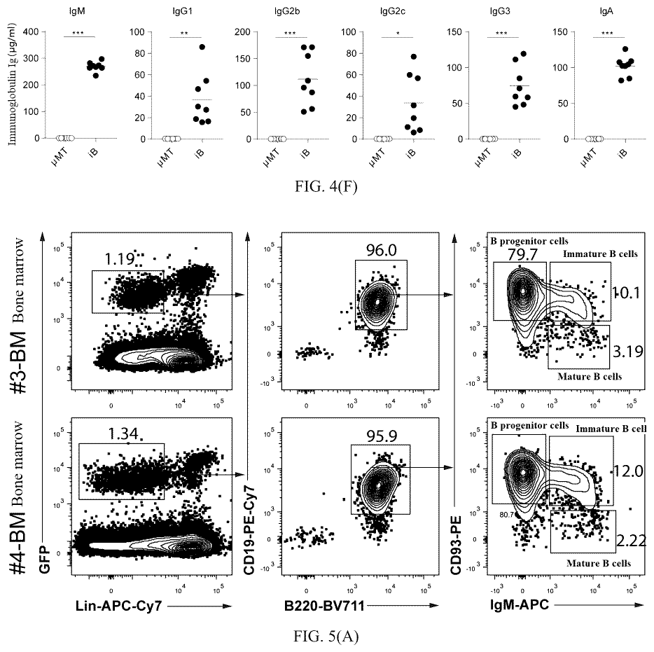

The transplantation strategy after the co-culture is shown in FIG. 4 (A). Dox was added to the induced hemogenic endothelial cells on the OP9-DL1 stromal cells to induce for 10 days to obtain the B-lineage seed cells. Then, the pluripotent stem cell-derived B-lineage seed cells were transplanted into 8 to 12-week-old B cell-deficient mice (μMT mice) via ophthalmic veins for the in vivo B lineage regeneration.

FIG. 4 (B) indicates that the B-lineage seed cells obtained after the co-culture of the iRUNX1-p2a-HOXA9-t2a-LHX2 pluripotent stem cell-derived induced hemogenic endothelial cells can form hematopoietic chimerism in various hematopoietic tissues and organs of the recipient μMT mice.

A flow cytometry analysis was performed on the recipient mice 6 weeks after the transplantation. The results show that in peripheral blood, bone marrow, spleens and lymph nodes, pluripotent stem cell-derived blood cells were mainly CD19⁺ cells, achieving an effect of effectively reconstituting B lymphocytes.

In this example, to confirm that GFP⁺ hematopoietic cells (mainly B cells) in the recipient mice were derived from the iRUNX1-p2a-HOXA9-t2a-LHX2 pluripotent stem cells at a genome level, primers were designed for PCR amplification and sequencing identification.

Flow cytometry sorting was performed on GFP⁺ cells derived from the bone marrow, the lymph nodes and the spleens, and the genomes were extracted for the PCR identification using the specific primers of the knock-in gene sequence (as shown in FIG. 4 (C)).

FIG. 4 (D) shows that the genomes of these cells had iRUNX1-p2a-HOXA9-t2a-LHX2 plasmid-derived sequences, confirming that the GFP⁺ blood cells (mainly the B cells) were derived from the iRUNX1-p2a-HOXA9-t2a-LHX2 pluripotent stem cells. Moreover, the sequencing results (as shown in FIG. 4 (E)) also prove this result.

Moreover, to verify whether the pluripotent stem cell-derived B cells had a function of secreting an antibody, contents of immunoglobulin in serums of unimmunized recipient mice were detected through ELISA assay 4 to 6 weeks after the transplantation.

As shown in FIG. 4 (F), compared with negative control μMT mice, various types of immunoglobulin including IgM, IgG1, IgG2b, IgG2c, IgG3 and IgA can be detected in serums of μMT recipient mice (iB mice) after the transplantation of the B-lineage seed cells, achieving an effect of reconstituting functional B lymphocytes.

### Example 5 Occurrence Process of B Lineage Regeneration

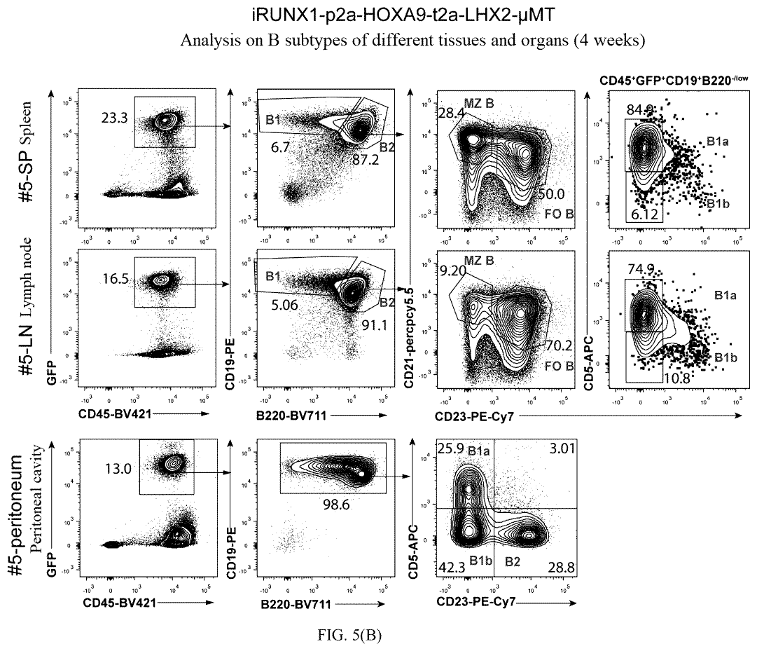

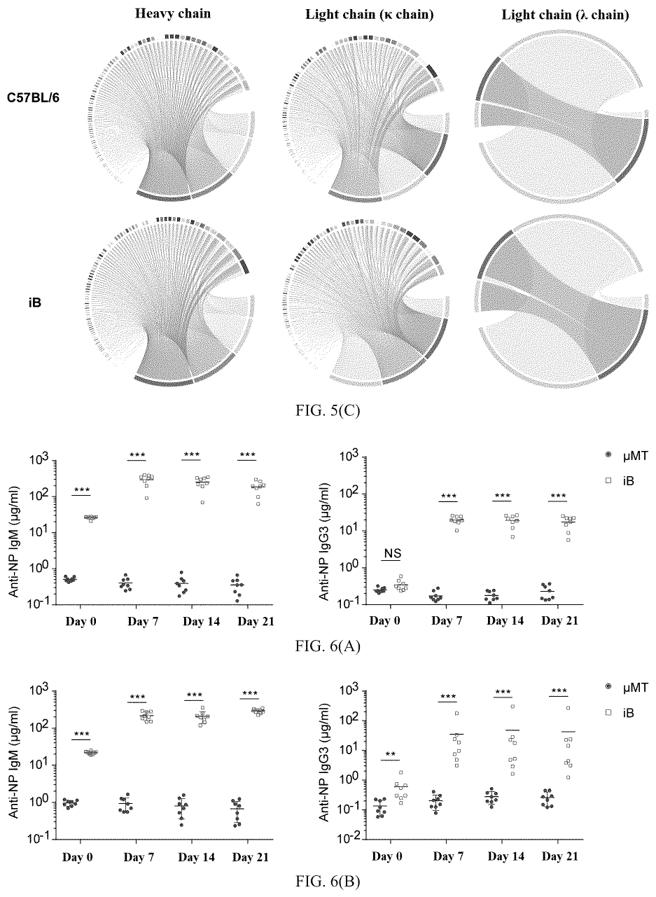

To further clarify the occurrence process of the iRUNX1-p2a-HOXA9-t2a-LHX2 pluripotent stem cell-derived B lineage, it was found through flow cytometry that as shown in FIG. 5 (A), the B lineage seed cells can regenerate pro/pre-B progenitor cells in the bone marrow and further develop into immature B cells and mature B cells.

FIG. 5 (B) shows that pluripotent stem cell-derived mature B1 cells (including Bla and Bib) and mature B2 cells (including FO B and MZ B) were present in the spleens, the lymph nodes and the peritoneal cavities.

Naive follicular B (FO B) cells in the spleens of the recipient mice (the iB mice) were sorted for B cell receptor (BCR) sequencing. As shown in FIG. 5 (C), the pluripotent stem cell-derived naive follicular B (FO B) cells had the diversity rearrangement of heavy chains and light chains, and the BCR diversity of the pluripotent stem cell-derived naive FO B was similar to the BCR diversity of naive FO B of C57BL/6 mice in a positive control group. This example confirms the normal development of the pluripotent stem cell-derived B lineage in the recipient mice.

### Example 6 Verification of Immune Function of B Cells

Whether the iRUNX1-p2a-HOXA9-t2a-LHX2 pluripotent stem cell-derived B cells can produce antigen-specific antibodies was further verified in this example.

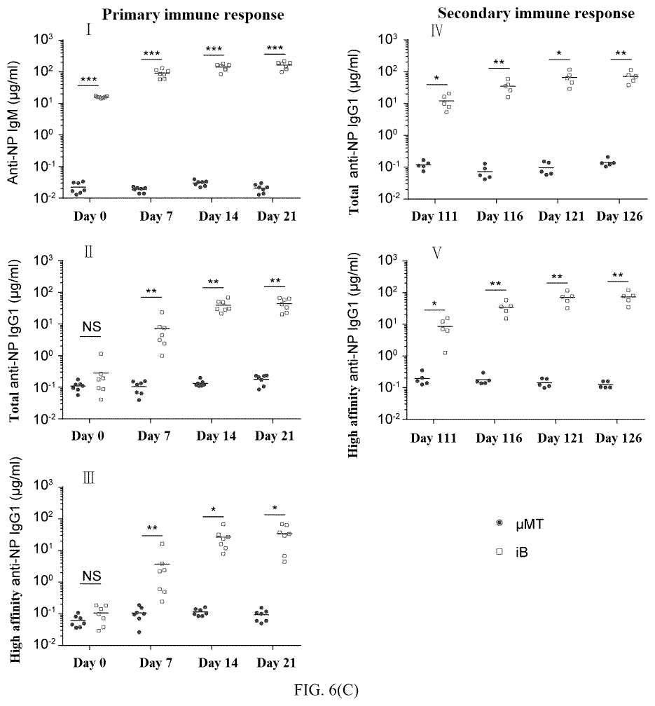

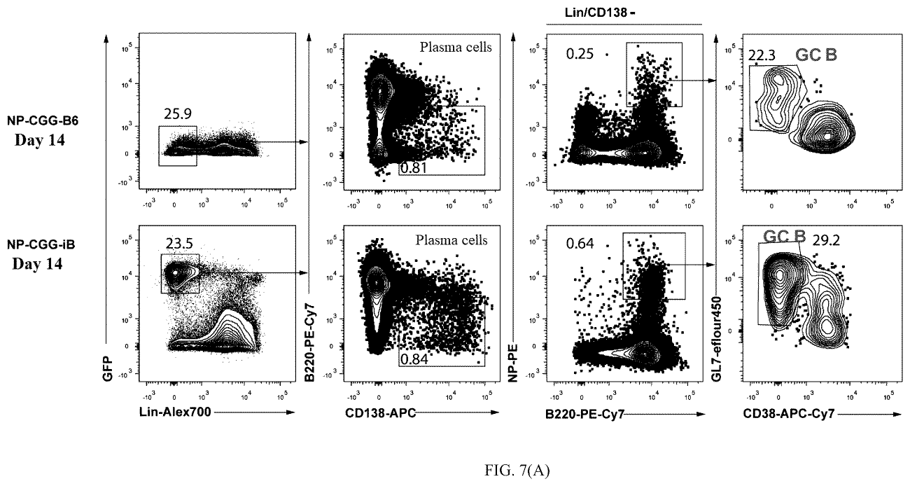

As shown in FIG. 6 (A), after the recipient mice (the iB mice) were immunized with T-independent-1 antigens (NP-LPS), the pluripotent stem cell-derived B cells can secrete antigen-specific (anti-NP) IgM and IgG3.

FIG. 6 (B) shows that after the recipient mice (the iB mice) were immunized with T-independent-2 antigens (NP-AECM-FICOLL), the pluripotent stem cell-derived B cells can secrete antigen-specific (anti-NP) IgM and IgG3.

FIG. 6 (C) shows that after the recipient mice (the iB mice) were immunized with T cell-dependent antigens (NP-CGG) the first time (FIGS. I, II and III) and the second time (FIGS. IV and V), the pluripotent stem cell-derived B cells can secrete antigen-specific (anti-NP) IgM and IgG1.

Therefore, this example confirms that the pluripotent stem cell-derived B cells can produce the specific antibodies for the specific antigens.

### Example 7 Adaptive Immune Response

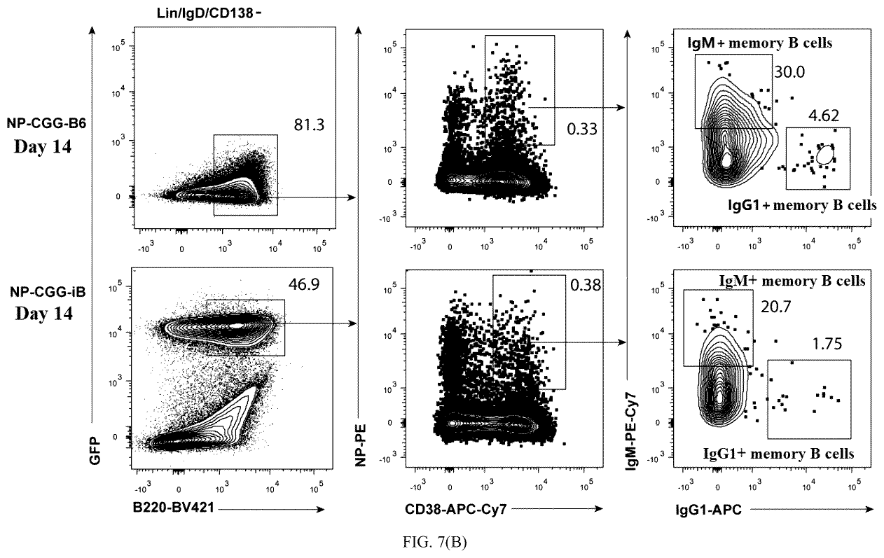

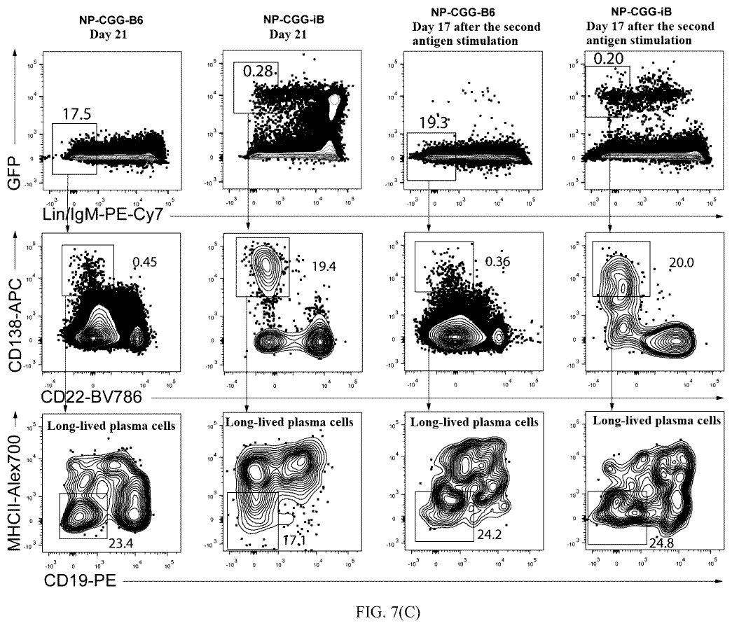

After the recipient mice (the iB mice) were immunized with the T cell-dependent antigens (NP-CGG), as shown in FIG. 7 (A), on day 14 after the immunization, the flow cytometry detection results show that the pluripotent stem cell-derived B cells in the spleens can form plasma cells and produce antigen-specific germinal center B (NP-specific GC B) cells.

FIG. 7 (B) shows that on day 14 after the immunization, IgM⁺ memory B cells can be detected in the spleens of the recipient mice and the pluripotent stem cell-derived B cells can undergo class switching to produce IgG1⁺ memory B cells.

FIG. 7 (C) shows that on the day 21 after the first antigen stimulation and on day 17 after the second antigen stimulation (the second antigen stimulation was performed on day 111 after the first antigen stimulation), long-lived plasma cells can be detected in the bone marrow of the recipient mice through flow cytometry.

Therefore, this example confirms that after the antigen stimulation, the pluripotent stem cell-derived B cells of the recipient mice can normally form the germinal center B (GC B) cells, the memory B cells and the long-lived plasma cells and can effectively participate in the adaptive immune response.

To conclude, in the present application, the vectors where exogenous RUNX1, HOXA9 and LHX2 are co-expressed are introduced into the pluripotent stem cells to successfully constitute the induced pluripotent stem cells where exogenous RUNX1, HOXA9 and LHX2 are co-expressed, and the pluripotent stem cells directionally differentiate into the B-lineage seed cells and develop into the B cells. The pluripotent stem cell-derived B cells obtained through the method of the present application not only have the normal functions, but also have no risk of tumorigenesis, and can be used for preparing drugs for enhancing an immune effect, preventing and/or treating an immunodeficiency, preventing and/or treating an infectious disease and preventing and/or treating a tumor, preparing a B cell vaccine and preparing a drug for a cell therapy that a B cell secretes a therapeutic protein.

The applicant states that the above are the specific examples of the present application and not intended to limit the protection scope of the present application. Those skilled in the art should understand that any changes or substitutions easily conceivable by those skilled in the art within the technical scope disclosed in the present application fall within the protection scope and the disclosed scope of the present application.

1. An expression vector, comprising a nucleotide sequence encoding a RUNX1 gene, a nucleotide sequence encoding a HOXA9 gene and a nucleotide sequence encoding an LHX2 gene.

2. The expression vector according to claim 1, wherein the nucleotide sequence encoding the RUNX1 gene, the nucleotide sequence encoding the HOXA9 gene and the nucleotide sequence encoding the LHX2 gene are linked in tandem by a nucleotide sequence encoding a 2A peptide.

3. The expression vector according to claim 2, wherein the 2A peptide comprises any one or a combination of at least two of T2A, P2A, E2A or F2A.

4. The expression vector according to claim 1, wherein in the expression vector, the nucleotide sequence encoding the RUNX1 gene, the nucleotide sequence encoding the HOXA9 gene and the nucleotide sequence encoding the LHX2 gene are linked in sequence, the nucleotide sequence encoding the RUNX1 gene and the nucleotide sequence encoding the HOXA9 gene are linked by a P2A nucleotide sequence, and the nucleotide sequence encoding the HOXA9 gene and the nucleotide sequence encoding the LHX2 gene are linked by a T2A nucleotide sequence.

5. A host cell, comprising the expression vector according to claim 1;
preferably, the host cell is a pluripotent stem cell comprising an induced pluripotent stem cell and/or an embryonic pluripotent stem cell line;
preferably, the pluripotent stem cell comprises a gene-edited induced pluripotent stem cell and/or embryonic pluripotent stem cell line.

6. A method for regenerating a humoral immunity system, comprising the following steps:
(1) integrating the expression vector according to claim 1 into a pluripotent stem cell and performing resistance cloning screening;
(2) directionally differentiating the pluripotent stem cell obtained in step (1) into an induced hemogenic endothelial cell;
(3) co-culturing the induced hemogenic endothelial cell in step (2) with a bone marrow stromal cell to obtain a B-lineage seed cell; and
(4) transferring the B-lineage seed cell in step (3) to an animal model and differentiating to produce a B cell.

7. The method according to claim 6, wherein a site where the expression vector is integrated into the pluripotent stem cell in step (1) comprises a ROSA26 site, an AAVS1 site, a CCR5 site, an H11 site, a COL1A1 site or a TIGRE site;
preferably, a method for the integration in step (1) comprises any one or a combination of at least two of homologous recombination, CRISPR/Cas9, TALEN, transfection or viral infection, preferably the homologous recombination;
preferably, hygromycin B is used for the resistance screening in step (1);
preferably, a method for the directional differentiation in step (2) is as follows: culturing the pluripotent stem cell using a DO medium, a D2.5 medium and a D6 medium in sequence to obtain the induced hemogenic endothelial cell;
preferably, the bone marrow stromal cell in step (3) comprises any one or a combination of at least two of an OP9-DL1 cell, an OP9-DL4 cell, an OP9 cell, an MS5 cell, an MS5-DL1 cell, an MS5-DL4 cell, an HS-5 cell, an HS-5-DL1 cell, an HS-5-DL4 cell, an MSC cell, an MSC-DL1 cell or an MSC-DL4 cell;
preferably, doxycycline is used for induction in a process of the co-culture in step (3);
preferably, a method for the co-culture in step (3) is as follows: co-culturing the induced hemogenic endothelial cell with the OP9-DL1 cell using a D11 medium to obtain the B-lineage seed cell.

8. The method according to claim 7, wherein the DO medium is a basal differentiation medium containing 3 to 8 ng/mL bone morphogenetic protein 4;
preferably, the D2.5 medium is a basal differentiation medium containing 3 to 8 ng/mL bone morphogenetic protein 4 and 3 to 8 ng/mL vascular endothelial growth factor;
preferably, the D6 medium is a basal differentiation medium containing 10 to 30 ng/mL interleukin 3, 10 to 30 ng/mL interleukin 6, 10 to 30 ng/mL stem cell factor, 10 to 30 ng/mL FMS-like tyrosine kinase 3 ligand and 1 to 2 μg/mL doxycycline;
preferably, the basal differentiation medium is an IMDM medium containing 10% to 20% fetal bovine serum, 180 to 220 μg/mL iron-saturated transferrin, 4×10⁻⁴ to 5×10⁻⁴ M thioglycerol, 1 to 3 mM GlutaMAX™-I additive and 30 to 70 μg/mL ascorbic acid;
preferably, the D11 medium is a-MEM medium containing 10 to 30 ng/mL interleukin 3, 10 to 30 ng/mL stem cell factor, 10 to 30 ng/mL FMS-like tyrosine kinase 3 ligand, 1 to 2 g/mL doxycycline, 10 to 20% fetal bovine serum, 180 to 220 μg/mL iron-saturated transferrin, 4×10⁻⁴ to 5×10⁻⁴ M thioglycerol, 1 to 3 mM GlutaMAX™-I additive and 30 to 70 μg/mL ascorbic acid.

9. The method according to claim 6, wherein the B cell produced through the differentiation in step (4) comprises a B220⁺ B cell and/or a CD19⁺ B cell;
preferably, the B cell produced through the differentiation comprises any one or a combination of at least two of a pro-B cell, a pre-B cell, a B1 cell, a B2 cell or a plasma cell;
preferably, the B1 cell comprises a B1a cell and/or a B1b cell;
preferably, the B2 cell is a follicular B cell and/or a marginal zone B cell.

10. A B-lineage seed cell or B cell prepared through the method according to claim 6.

11. A pharmaceutical composition, comprising any one or a combination of at least two of the expression vectors according to claim 1.

12. A method for enhancing an immune response in a subject comprising administering the pharmaceutical composition according to claim 11 to a subject in need thereof;
preferably, the drug for enhancing the immune response comprises a drug for enhancing a B cell immune response and/or a T cell immune response.

13. The method according to claim 12, the pharmaceutical composition preventing and/or treating a disease,
preferably, the pharmaceutical composition preventing and/or treating a B cell immunodeficiency, an infectious disease, and a tumor.

14. The method according to claim 12, wherein the pharmaceutical composition provides B cell immunotherapy for treating a tumor,
preferably, the B cell secretes a therapeutic protein comprises a drug for preventing and/or treating an autoimmune disease and a genetically inherited disease,
preferably, the genetically inherited disease comprises any one or a combination of at least two of hemophilia, lysosomal storage disease, hypophosphatasia or phenylketonuria.

15. The method according to claim 12, wherein the pharmaceutical composition is a B cell vaccine or a drug for a cell therapy in which a B cell secretes a therapeutic protein;
preferably, the therapeutic protein secreted by the B cell comprises an antibody.

16. A pharmaceutical composition, comprising the host cell according to claim 5.

17. A pharmaceutical composition, comprising the B-lineage seed cell or B cell according to claim 10.

18. A pharmaceutical composition according to claim 11, wherein the pharmaceutical composition further comprises a pharmaceutically acceptable adjuvant.
